(19) United States
(12) Patent Application Publication
(10) Pub. No.: US 2019/0015349 A1
(43) Pub. Date: Jan. 17, 2019

(54) MUCUS-PENETRATING BUDESONIDE NANOSUSPENSION ENEMA FOR LOCAL TREATMENT OF INFLAMMATORY BOWEL DISEASE

(71) Applicant: The Johns Hopkins University, Baltimore, MD (US)

(72) Inventors: Justin Hanes, Baltimore, MD (US); Laura M. Ensign, Towson, MD (US)

(21) Appl. No.: 16/036,819

(22) Filed: Jul. 16, 2018

Related U.S. Application Data
(60) Provisional application No. 62/532,883, filed on Jul. 14, 2017.

(51) Int. Cl. A61K 9/51; A61K 31/58; A61P 1/00; A61K 9/00; A61K 9/02; A61K 49/00

(52) U.S. Cl. CPC A61K 9/5146; A61K 31/58; A61K 49/0093; A61K 9/0031; A61K 9/025; A61P 1/00

(57) ABSTRACT

Inflammatory bowel disease (IBD) is a chronic inflammatory gastrointestinal disorder that affects more than 1 million individuals in the USA. Described herein are nanoparticle mucus penetrating formulations for administration of drugs for improved mucosal distribution and tissue penetration such as in the treatment of IBD.

### CROSS-REFERENCE TO RELATED APPLICATIONS

This application claims priority to U.S. Ser. No. 62/532,883, filed Jul. 14, 2017 by Justin Hanes and Laura M. Ensign for “Mucus-Penetrating Budesonide Nanosuspension Enema for Local Treatment of Inflammatory Bowel Disease”, the disclosure of which are hereby incorporated herein by reference in their entirety.

### STATEMENT REGARDING FEDERALLY SPONSORED RESEARCH

This invention was made with Government support under Grant No. R21/R33AI079740 and Grant No. R01DK107806 awarded by the National Institutes of Health. The Government has certain rights in the invention.

### BACKGROUND OF THE INVENTION

Inflammatory bowel disease (IBD) is a chronic inflammatory gastrointestinal disorder that affects more than 1 million individuals in the USA. Local therapy with enema formulations, such as micronized budesonide (Entocort®), is a common strategy for treating patients with distally active IBD. These formulations utilized micronized particulates that are too large to effectively penetrate colorectal mucus, limiting the extent of drug delivery to affected tissues prior to clearance.

This problem is equally applicable to other drugs which are administered orally to treat gastrointestinal disorders.

It is therefore an object of the present invention to provide improved formulations for oral administration of drugs that must be delivered to the epithelium through the gastric mucosa.

### SUMMARY OF THE INVENTION

A budesonide nanosuspension (NS) with the appropriate surface coating and size to enhance penetration of colorectal mucus and ulcerated colorectal tissues has been developed. The surface coating is formed of mucosal penetrating water soluble or hydrophilic polymers, most typically polyalkylene oxide polymers or copolymers. The preferred coating is Pluronic F127. The nano-size and muco-inert coating provides enhanced local delivery of budesonide, and thus, a more significant impact on local colorectal tissue inflammation. This technology is applicable to other drugs that are administered orally and need to penetrate the intestinal mucosa.

As demonstrated by the example, model fluorescent polystyrene (PS) particles ˜200 nm in size with a muco-inert Pluronic F127 coating provide enhanced mucosal distribution and tissue penetration in mice with trinitrobenzenesulfonic acid (TNBS)-induced IBD compared to model 2 μm PS particles coated with polyvinylpyrollidone (PVP) to mimic the clinical micronized budesonide formulation. A wet-milling process was used to make a budesonide NS formulation with a muco-inert Pluronic F127 coating (particle size ˜230 nm), as well as a budesonide microsuspension (MS) coated with PVP (particle size ˜2 μm).

In an acute TNBS mouse model of IBD, daily budesonide NS enema treatment resulted in a significant reduction in the macroscopic (decreased colon weight) and microscopic (histology score) symptoms of IBD compared to untreated controls or mice treated daily with the budesonide MS enema. Further, the budesonide NS enema treated mice had a significantly reduced number of inflammatory macrophages and IL-β producing CD11b+ cells in colon tissue compared to untreated controls or mice treated with the budesonide MS enema.

### BRIEF DESCRIPTION OF THE DRAWINGS

FIG. 1A-1D are graphs showing optimization, characterization and in vitro anti-inflammatory activity of budesonide NS. Various parameters of wet-milling process such as size of zirconium oxide beads (FIG. 1A); budesonide to bead mass ratio (1B); Pluronic F127 concentration(1C) and wet-milling time (1D)were optimized to obtain Pluronic F127 coated budesonide NS in the size range appropriate for penetration of mouse colorectal mucus (˜222 nm and polydispersity index ≤0.2).

FIG. 2 is a graph of In vitro anti-inflammatory activity of budesonide NS was evaluated to assess the impact of wet-milling process on drug activity. RAW264.7 cells were activated with LPS (0.5 μg/ml) for 5 hours in the presence or absence of 1 μM of budesonide solution (SOL), MS, or NS and the production of the pro-form of IL-1β from CD11b+ RAW264.7 cells was measured using flow cytometry. Budesonide SOL, MS and NS were able to reduce the production of the pro-form of IL-1β in LPS activated RAW264.7 cells compared to control (CTRL). Data are expressed as mean ±SEM (n≥3). *p<0.05, **p<0.01.

FIGS. 3A-3C are graphs showing treatment with the budesonide NS enema provides significant reductions in macroscopic and microscopic symptoms in mice with TNBS-induced colitis (3A). Mice treated with budesonide MS (TNBS+MS) or NS (TNBS+NS) enema showed a significant recovery in body weight compared to untreated (TNBS) animals (3B). Only the budesonide NS treated animals showed significantly lower colon weight compared to untreated animals (n≥15 per group) (3C). Quantitative histopathological scoring based on mucosal damage, destruction of cellular architecture, infiltration and epithelial regeneration, and percent colon involved demonstrated a significant reduction in disease with budesonide NS enema treatment compared to untreated mice and MS treated mice (n=5 per group). Data are expressed as mean ±SEM; *p<0.05, **p<0.01

FIGS. 4A-4C are graphs showing only budesonide NS significantly lowered the number of viable inflammatory macrophages, a cell type implicated in the pathogenesis of IBD, in the colon lamina propria. (4A) Treatment with budesonide MS and NS significantly reduced the number of infiltrating neutrophils (Ly6G⁺/CD45⁺ cells; n≥5 per group), and (4B) only budesonide NS showed reduction in inflammatory macrophages (Ly6C^{high+}/CD11b⁺/CD45⁺ cells; n≥7 per group) and (4C) reduction in the production of the pro-form of inflammatory cytokine IL-1β from the CD11b+ cells (n≥4 per group). Data are expressed as mean ±SEM. *p<0.05, **p<0.01, ***p<0.001.

FIGS. 5A-5D are graphs showing in vitro anti-inflammatory activity of budesonide MS and NS. MLN cells and splenocytes were isolated from C57BL/6 mice and activated with LPS (1 μg/ml), phorbol myristyl acetate (PMA; 50 ng/ml) and ionomycin (Ion; 500 ng/ml) for 5 hours in the presence or absence of 1 μM of budesonide MS or NS and the mean fluorescence intensity (MFI) of (A) & (C) TNF-α gated on CD11b+ cells and (B) & (D) interferon-γ gated on CD4+ T cells was measured using flow cytometry. Budesonide MS and NS were able to reduce the MFI of inflammatory cytokines in activated MLN cells and splenocytes. Data are expressed as mean ±SEM (n≥3). **p<0.01, ***p<0.001.

FIGS. 6A-6C are graphs showing budesonide MS and NS modulate the colonic adaptive immune system in the TNBS-induced colitis model. Treatment with budesonide MS and NS reduced the number of CD4 T cells (CD4⁺/CD45⁺) (6A) and CD8 T cells (CD8⁺/CD45⁺) (6B) and significantly reduced the number of B cells (CD19⁺/CD45⁺) in the colon lamina propria 6C). Data are expressed as mean ±SEM (n≥4 per group). *p<0.05.

### DETAILED DESCRIPTION OF THE INVENTION

Although oral administration is often considered the most acceptable mode of drug administration, topical drug administration results in much higher local drug levels with lower doses, as well as reduced systemic drug exposure. This is particularly true for irritable bowel disease (“IBD”) drugs like budesonide, which undergoes extensive first-pass metabolism, and has undesirable systemic side effects. Topical enema therapy is more effective for IBD patients with disease affecting only the distal colorectum, and even for patients with additional diffuse active disease, with the combination of both an enema and oral medication being more effective than oral therapy alone.

Numerous efforts for formulating nanoparticles that can be dosed orally, but transit to the colorectum to deliver drug locally, have been described. There are some limitations to this type of approach, which typically requires the use of an enteric polymer coating to minimize drug release in other parts of the GI tract. The pH changes throughout the GI tract are an attractive attribute for using pH to modulate particle degradation and drug release, but there has been preclinical and clinical evidence demonstrating interpatient variability that leads to premature degradation, or no degradation at all. Particularly in diseased states like IBD, the normal physiology, including pH, is altered. Further, loading drugs into nanoparticles, whether polymeric or lipid-based, often results in low drug loadings (<0.1-6%), meaning that the bulk of the administered material is polymer or other excipients. In contrast, the NS approach described here is composed of pure budesonide milled down to nano-size and coated with FDA generally regarded as safe (GRAS) surfactant, preferably polyoxyethylene or polyethylene glycol block copolymers, most preferably Pluronic F127.

Micro- and nanosuspensions are fluid formulations that contain water insoluble drugs that have been processed through bottom-up or top-down methods to form semi-stable suspensions of amorphous or crystalline drug particles. The approach is intended to increase the surface area-to-volume ratio for improved dissolution, and has been thoroughly explored and developed to increase oral bioavailability of poorly bioavailable drugs. Micronization is also a strategy employed for clinically available enema formulations (budesonide and mesalamine) for local treatment of ulcerative colitis. However, large microparticulates are unlikely to penetrate the colorectal mucus barrier and be delivered to the affected tissues. NS are more efficacious for IBD enema therapies.

Prior descriptions of budesonide NS include the use of a high-pressure homogenization apparatus to produce large scale batches (300 mL) for pulmonary administration, though the particle size achieved was 500-600 nm. In another report, budesonide NS were produced via a bench top wet milling method using glass beads. However, the method required 24 h of milling and resulted in a budesonide NS with ˜400 nm average particulate size. In contrast, the modified wet milling described here using commercially available tissue homogenization equipment (TissueLyser LT) and inert zirconium oxide beads u henables transformation of budesonide in to NS formulation of the appropriate size (˜225 nm, although there is a range of particle sizes that can be used, from 150 to 600, more preferably from 200 to 450, most preferably between 200 and 300 nm) and surface characteristics (Pluronic F127 coated, −2 mV) for effective mucus penetration and improved colorectal distribution.

Intestinal inflammation leads to mucus hypersecretion and a reduction in barrier function, which has been demonstrated to have an impact on particle distribution and accumulation. A reduction in steric barrier properties in the TNBS-induced colitis model was previously observed, though adhesive interactions with mucoadhesive nanoparticles were maintained. Others have similarly found increased accumulation of orally administered nanoparticles in inflamed areas of the intestines, presumably due to increased mucus production and a thicker mucus layer, which facilitates particle entrapment and adhesion. However, there have been conflicting reports for how size impacts accumulation of particles at the inflamed mucosa; in several reports using animal models, smaller particle size lead to increased accumulation, whereas a clinical study suggested that microparticles accumulated more in areas of severe disease with ulcerous lesions compared to nanoparticles. The surface properties and core composition of the particles used in the aforementioned studies varied, and thus, one cannot determine the impact of particle size, surface properties, and particle composition on particle uptake and drug delivery in IBD patients. Further, the increased accumulation of particles, particularly mucoadhesive particles, at inflamed mucosa makes determining discrete drug levels in colorectal cells and tissue technically challenging. The adherent mucus layer may be particularly challenging to remove, and differences in the efficiency of removing various sized particles by extensive washing could be a result of differences in penetration depth or differences in cell uptake. It is particularly challenging to remove the luminal contents to measure drug absorption into tissue with drug-loaded particulate formulations in preclinical IBD models, and thus, particle localization studies and/or efficacy studies are the norm for assessing drug delivery efficiency.

### I. Definitions

“Inflammatory bowel disease (IBD)”, as used herein, generally refer to chronic inflammatory diseases that affect the gastrointestinal tract, further divided into ulcerative colitis (UC) and Crohn's disease (CD). IBD is generally characterized by immune system dysregulation, mucosal inflammation, and impaired integrity of the epithelial barrier.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual. “Hydrophilic,” as used herein, refers to the property of having affinity for water. For example, hydrophilic polymers (or hydrophilic polymers) are polymers (or polymers) which are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer), the more that polymer (or polymer) tends to not dissolve in, not mix with, or not be wetted by water.

Hydrophilicity and hydrophobicity can be spoken of in relative terms, such as, but not limited to, a spectrum of hydrophilicity/hydrophobicity within a group of polymers or polymers. In some embodiments wherein two or more polymers are being discussed, the term “hydrophobic polymer” can be defined based on the polymer's relative hydrophobicity when compared to another, more hydrophilic polymer. “Microparticle,” as used herein, generally refers to a particle having a diameter, such as an average diameter, from about 1 micron to about 100 microns, preferably from about 1 micron to about 50 microns, more preferably from about 1 to about 30 microns. The microparticles can have any shape.

“Nanoparticle,” as used herein, generally refers to a structure of any shape having a diameter from about 1 nm up to, but not including, about 1 micron, more preferably from about 5 nm to about 500 nm. Nanoparticles having a spherical shape are generally referred to as “nanospheres”. Non-limiting examples of nanoparticles include soft nanoparticles, e.g., micelles, colloids, liposomes, vesicles, nanodroplets nano-structured hydrogel, nanocrystals, and nanosuspension. Soft nanoparticles generally dissolve or dissemble to release agents.

The term “nanosuspension,” as used herein refers to a submicron colloidal, dispersion of drug particles.

As used herein, the term “treating” includes inhibiting, alleviating, preventing or eliminating one or more symptoms or side effects associated with the disease, condition, or disorder being treated.

The term “reduce”, “inhibit”, “alleviate” or “decrease” are used relative to a control. One of skill in the art would readily identify the appropriate control to use for each experiment. For example a decreased response in a subject or cell treated with a compound is compared to a response in subject or cell that is not treated with the compound.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effect of the effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

“Excipient” is used herein to include any other compound that can be included in the formulation that is not a therapeutically or biologically active compound. As such, an excipient should be pharmaceutically or biologically acceptable and non-toxic to the subject when administered by the intended route.

The term “POLOXAMER” as used herein is a trademark referring to nonionic triblock copolymers composed of a central hydrophobic chain of polyoxypropylene (poly(propylene oxide)) flanked by two hydrophilic chains of polyoxyethylene (poly(ethylene oxide)).

The term “Hypotonic enema carrier/vehicle” delivers drugs advectively to the colon epithelium by the bulk flow of water (advection) and is nontoxic. [print claims, highlight words that you think are key to understand and describe the invention, provide here definitions of those words, also define any words that are important but ambiguous, s.a. “about”]

The term “osmolarity”, as generally used herein, refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na⁺ and Cl⁻ in solution and thus provides 2 moles of dissolved components per 1 mole of dissolved NaCl in solution. Physiological osmolarity is typically in the range of about 280 to about 310 mOsm/L.

The term “tonicity”, as generally used herein, refers to the osmotic pressure gradient resulting from the separation of two solutions by a semi-permeable membrane. In particular, tonicity is used to describe the osmotic pressure created across a cell membrane when a cell is exposed to an external solution. Solutes that readily cross the cellular membrane contribute minimally to the final osmotic pressure gradient. In contrast, those dissolved species that do not cross the cell membrane, “impermeable solutes”, will contribute to osmotic pressure differences and thus tonicity. The term “hypertonic”, as generally used herein, refers to a solution with a higher concentration of impermeable solutes than is present on the inside of the cell. When a cell is immersed into a hypertonic solution, water will flow out of the cell, concentrating the impermeable solutes inside the cell until it becomes equal to the concentration of impermeable solutes outside the cell. The term “hypotonic”, as generally used herein, refers to a solution with a lower concentration of impermeable solutes than is present inside of the cell. When a cell is immersed into a hypotonic solution, water will flow into the cell, diluting the concentration of impermeable solutes inside the cell until it becomes equal to the concentration of impermeable solutes outside the cell. The term “isotonic”, as generally used herein, refers to a solution wherein the osmotic pressure gradient across the cell membrane is essentially balanced and no water flows into or out of the cell. The same meanings for tonicity apply for water flow through intestinal epithelia; hypertonic solutions cause water to flow into the lumen, whereas hypotonic solutions cause water to flow out of the lumen. Tonicity depends on the permeability properties of the cell or epithelium to different solutes, whereas osmolarity depends only on the total concentration of all solutes.

### II. Composition

To achieve effective topical treatment for inflammation in the colorectum, sustained, therapeutic levels of drug must be delivered to the affected tissues and cells. Although the colorectum is relatively accessible by enema administration, additional barriers to effective local drug delivery exist. The mucus layers coating the colorectal epithelial surface are a major barrier that limits colorectal drug distribution and absorption into the local tissue. Frontline enema formulations for a water insoluble drug like budesonide contain micronized drug particles. However, it was discovered that microparticles are too large to penetrate the protective mucus mesh lining the colorectum and, thus, are sterically restricted from reaching the epithelial surface, facilitating rapid clearance. In contrast, it was discovered that nanoparticles that are both (i) small enough to pass through the pores in the mucus mesh, and (ii) non-adhesive to mucus (mucus-penetrating particles, MPP), provide rapid, highly uniform epithelial distribution in the colorectum following enema administration in a hypotonic enema vehicle. Hypotonic vehicles cause rapid, osmosis-driven, advective delivery of non-adhesive MPP through mucus, and right up against the epithelial surface. Further, MPP administered in a hypotonic enema vehicle entered ulcerated tissues in the colorectum in a mouse model of 2,4,6-trinitrobenzenesulfonic acid (TNBS)-induced IBD more efficiently than microparticles or nanoparticles that were adhesive to mucus.

The design of budesonide nanosuspensions (NS) containing drug particles of the appropriate size and formulated with muco-inert coatings for improved mucosal distribution and tissue penetration in IBD. It was demonstrated that budesonide NS provide improved colorectal tissue levels of budesonide compared to budesonide microsuspensions (MS) with minimal systemic exposure. The budesonide formulations were further compared for efficacy in the TNBS-induced mouse model of IBD, because (i) the inflammatory injury is localized to the colorectum, which is optimal for enema delivery, and (ii) budesonide was previously shown to be efficacious in the TNBS mouse model of IBD. The improved colorectal drug delivery and efficacy provided by the muco-inert budesonide NS shows the potential for clinical utility.

A. Polymer

Biocompatible polymers are generally used to coat the drug nanoparticles. In one embodiment, the biocompatible polymer(s) is biodegradable or bioabsorbable.

Exemplary polymers to coat the drug nanoparticles (e.g., capable of dissolution to release agent) include, but are not limited to, other polyamino acids; cyclodextrin-containing polymers, cationic cyclodextrin-containing polymers; polymers prepared from lactones such as poly(caprolactone) (PCL); polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), and blends thereof, polyalkyl cyanoacralate, polyurethanes, poly(valeric acid), and poly-L-glutamic acid; hydroxypropyl methacrylate (HPMA); polyanhydrides; other polyesters; polyorthoesters; poly(ester amides); polyamides; poly(ester ethers); polycarbonates; polyalkylenes such as polyethylene and polypropylene; polyalkylene glycols such as poly(ethylene glycol) (PEG) and polyalkylene oxides (PEO), and block copolymers thereof such as polyoxyalkylene oxide (“PLURONICS®” or block copolymers containing PEG where PEG has a molecular weight of any values within the range of 300 Daltons to 1 MDa); polyalkylene terephthalates such as poly(ethylene terephthalate); ethylene vinyl acetate polymer (EVA); polyvinyl alcohols (PVA); polyvinyl ethers; polyvinyl esters such as poly(vinyl acetate); polyvinyl halides such as poly(vinyl chloride) (PVC), polyvinylpyrrolidone; polysiloxanes; polystyrene (PS); and celluloses including alkyl celluloses, hydroxyalkyl celluloses, cellulose ethers, cellulose esters, nitro celluloses, hydroxypropylcellulose, and carboxymethylcellulose; polymers of acrylic acids including poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”); polydioxanone and its copolymers; polyhydroxyalkanoates; polypropylene fumarate; polyoxymethylene; poloxamers; poly(butyric acid); trimethylene carbonate; and polyphosphazenes.

Examples of natural polymers include proteins such as albumin, collagen, gelatin and prolamines, for example, zein, and polysaccharides such as alginate.

Copolymers of the above, such as random, block, or graft copolymers, or blends of the polymers listed above can also be used.

In some embodiments where polyalkylene glycol (e.g., PEG) is used as in the polymer to coat the particles for delivery, PEG surface density may be controlled by varying the amount of PEG in the polymer composition or by mixing a blend of pegylated polymer component and non-pegylated polymer component. The density of PEG or polyalkylene glycol on the surface of the particles may be evaluated using several techniques. For example, nuclear magnetic resonance (NMR), both qualitatively and quantitatively (PEG peak typically observed ˜3.65 ppm). When particles are dispersed within the NMR solvent D₂O, only the surface PEG, not the PEG embedded within the core, can be directly detected by NMR. Therefore, NMR provides a means for directly measure the surface density of PEG. In some forms, delivery to bladder tissue shows an improved drug absorption and retention when lower amount or no PEG is used. In these forms, PEG density is below approximately 20, 10, or five PEG chains/100 nm², or the mass of PEG in the particle excluding the active agent is less than 70%, 50%, 30%, 25%, or 10%.

In some embodiments, the particles possess a ζ-potential of between about 20 mV and about −20 mV, between about 10 mV and about −10 mV, between about 2 mV and about −2 mV.

B. Therapeutic, Prophylactic, and Diagnostic Agents

A wide range of agents may be in the particles to be delivered. These may be proteins or peptides, sugars or carbohydrate, nucleic acids or oligonucleotides, lipids, small molecules, or combinations thereof. In some embodiments, the particles have encapsulated therein, dispersed therein, and/or covalently or non-covalently associate with the surface one or more agents.

Therapeutic Agents

Exemplary classes of therapeutic agents include, but are not limited to, analgesics, anti-inflammatory drugs such as budesonide, anti-proliferatives such as anti-cancer agent, anti-infectious agents such as antibacterial agents and antifungal agents, antihistamines, corticosteroids, dopaminergics, and muscle relaxants.

Diagnostic Agents

Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Examples of other suitable contrast agents include gases or gas emitting compounds, which are radioopaque. Nanoparticles can further include agents useful for determining the location of administered particles. Agents useful for this purpose include fluorescent tags, radionuclides and contrast agents. These agents can also be used prophylactically.

C. Formulations

Liquid Formulations

Liquid formulations contain one or more assembled particles suspended in a liquid pharmaceutical carrier.

Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Formulations may be prepared using one or more pharmaceutically acceptable excipients, including diluents, preservatives, binders, lubricants, disintegrators, swelling agents, fillers, stabilizers, and combinations thereof. Liquid formulations may also contain minor amounts of polymers, surfactants, or other excipients well known to those of the art. In this context, “minor amounts” means no excipients are present that might adversely affect the delivery of assembled gel compositions to targeted tissues, e.g. through circulation.

Dry Powder Formulations and Kit

In some forms, the gelators, stabilizing agents, and optionally one or more therapeutic, prophylactic, and diagnostic agents are formulated in dry powder forms as finely divided solid formulations. The dry powder components can be stored in separate containers, or mixed at specific ratios and stored. In some embodiments, suitable aqueous and organic solvents are included in additional containers. In some embodiments, dry powder components, one or more solvents, and instructions on procedures to mix and prepare assembled nanostructures are included in a kit. Alternatively, stabilized, assembled particles, nanoparticles or bulk gel thereof are dried via vacuum-drying or freeze-drying, and suitable pharmaceutical liquid carrier can be added to rehydrate and suspend the assembled nanostructures or gel compositions upon use.

Dry powder formulations are typically prepared by blending one or more gelators, stabilizing agents, or active agents with one or more pharmaceutically acceptable carriers. Pharmaceutical carrier may include one or more dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride.

The dry powder formulations can be suspended in the liquid formulations to form assembled particles or nanoparticles thereof, and administered systemically or regionally using methods known in the art for the delivery of liquid formulations.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzyl peroxide, benzethonium chloride, benzyl alcohol, cetylpyridinium chloride, chlorobutanol, phenol, phenylethyl alcohol, and thimerosal.

Formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules.

Hypotonic Carriers/Vehicles

The compositions may include a hypotonic carrier. The hypotonic carrier will typically be a biocompatible carrier that preferably causes little to no signs of irritation when administered to human subjects. The carrier can be naturally occurring or non-naturally occurring including both synthetic and semi-synthetic carriers. Preferred carriers are sodium-based. Other solutions, including sugar-based (e.g. glucose, mannitol) solutions and various buffers (phosphate-buffers, tris-buffers, HEPES), may also be used.

When hypotonic solutions are applied to an epithelial surface, water flows out of the lumen, into cells and across the epithelium. This can cause swelling of the epithelial cells. In some cases, when the osmotic pressure difference is too large, the epithelial cells may burst, causing tissue irritation or disruption of the epithelial lining.

Advective transport of solutes is dominated by the bulk flow of a fluid, as in a solution passing through a filter. Since the colon absorbs water to dry the feces, fluid absorption by the colorectum can transport drugs advectively to the epithelium with great rapidity, much faster than by diffusion, and can move solutes through the unstirred layer of mucus adhering to the colonic epithelium. This distributes solutes to the entire colorectal surface, and if the formulation composition selectively improves tissue absorption rather than systemic absorption, minimizes systemic toxic side effects. The formulation for drug delivery markedly improves the uniformity of distribution of drugs over the epithelial surface. The formulations are particularly effective for delivery of microbicides for preventing rectal HIV transmission, as well as therapeutic drugs for the colon.

An absorption-inducing (hypotonic) enema delivers drugs advectively to the colon epithelium by the bulk flow of water (advection) and is nontoxic. This was demonstrated by advective delivery of a small hydrophilic drug in solution (tenofovir, a candidate microbicide for blocking HIV). The absorption-inducing hypotonic enema formulations caused free drug to be transported rapidly to the epithelial surface, unimpeded by the unstirred mucus barrier coating the epithelium. Moreover, advective transport delivered free drug deep into the colorectal folds to reach virtually the entire colorectal epithelial surface. In contrast, secretion-inducing (hypertonic) enema formulations markedly reduced drug uptake, and caused free drug to be expelled from the colon. Enemas induced rapid absorption even when sodium chloride (NaCl) was moderately hyperosmolal with respect to blood (˜500 vs ˜300 mOsm), presumably because sodium is actively pumped out of the lumen of the colon.

Hypotonic solution refers to a solution that contains less impermeable solutes compared to the cytoplasm of the cell. Examples of hypotonic solutions include, but are not limited to, Tris[hydroxylmethyl]-aminomethane hydrochloride (Tris-HCl, 10-100 mM, pH. 6-8), (4-(2-hydroxyethyl)-1-piperazineethanesulfonic acid (HEPES, 10-100 mM, pH 6-8) and dilute solutions of PBS, such as a solution containing 0.2 grams KCl, 0.2 grams KH₂PO₄, 8 grams NaCl, and 2.16 grams Na₂HPO₄*7H₂O in 1000 ml H₂O, and dilute solutions of normal saline (typically containing 0.9% NaCl).

The hypotonic carrier usually contains water as the major component. The hypotonic carrier can be water, although mixtures of water and a water-miscible organic solvent can also be used. Suitable water-miscible organic solvents include alcohols, such as ethanol, isopropanol; ketones, such as acetone; ethers, such as dioxane and esters such as ethyl acetate.

The hypotonic carrier can be water containing one or more tonicity modifying excipients. Sodium chloride is the excipient that is most frequently used to adjust tonicity of a solution. Other excipients used to adjust the tonicity of solutions include glucose, mannitol, glycerol, propylene glycol and sodium sulphate. Tonicity modifying excipients can include pharmaceutically acceptable salts such as sodium chloride, sodium sulfate, or potassium chloride. Other excipients used to adjust tonicity can include glucose, mannitol, glycerol, or propylene glycol.

The tonicity of a formulation varies for different cells and mucosal surfaces; it also depends on whether or not the cell or epithelium actively transports solutes and ions; e.g. it has been found that the isotonic point in the vagina for sodium-based solutions is about 300 mOsm/L, similar to the osmolarity of serum, but in the colorectum, it is significantly higher, about 450 mOsm/L (presumable because the colorectum actively transports sodium ions out of the lumen). In some embodiments the solution has a tonicity from 50 mOsm/L to 280 mOsm/L, from 100 mOsm/L to 280 mOsm/L, from 150 mOsm/L to 250 mOsm/L, from 200 mOsm/L to 250 mOsm/L, from 220 mOsm/L to 250 mOsm/L, from 220 mOsm/L to 260 mOsm/L, from 220 mOsm/L to 270 mOsm/L, or from 220 mOsm/L to 280 mOsm/L.

The hypotonic carrier can include one or more pharmaceutically acceptable acids, one or more pharmaceutically acceptable bases, or salts thereof. Pharmaceutically acceptable acids include hydrobromic, hydrochloric, and sulphuric acids, and organic acids, such as lactic acid, methanesulphonic acids, tartaric acids, and malcic acids. Pharmaceutically acceptable bases include alkali metal (e.g. sodium or potassium) and alkali earth metal (e.g. calcium or magnesium) hydroxides and organic bases such as pharmaceutically acceptable amines. The hypotonic carrier can include pharmaceutically acceptable buffers such as citrate buffers or phosphate buffers.

### III. Methods of Treatment

The formulation described herein can be used for the administration of any enterally delivered drug, especially those requiring penetration of the mucosa in the colon.

In a preferred embodiment, the formulation is administered as an enema, orally, or via enteric coated capsule which releases after passage through the stomach and within the small and large intestines.

Many diseases and disorders in which the drug is administered via the intestinal mucosa and into the underlying tissue can be treated with drug formulated as described herein. Representative disorders include inflammatory bowel disease, colitis, spastic colon, and Crohn's disease (CD). The formulation can also be used to treat gut cramps, diarrhea, constipation, and some types of chemotherapy, especially those resulting in mucositis.

Inflammatory bowel disease (IBD) is a term that refers to chronic inflammatory diseases that affect the gastrointestinal tract, further divided into ulcerative colitis (UC) and Crohn's disease (CD). IBD is generally characterized by immune system dysregulation, mucosal inflammation, and impaired integrity of the epithelial barrier. Anti-inflammatory drugs, such as steroids, are commonly used to treat IBD symptoms, repair mucosal tissues, and induce remission. The current frontline steroid therapy is budesonide, which has low bioavailability and undergoes extensive first-pass metabolism when taken orally, but is considered to be more effective than other common steroids when dosed topically. For two-thirds of UC patients and for patients with more diffuse disease that also affects the distal colorectum, topical enema therapy is used.

In the preferred embodiment, the formulation is administered as a suspension in an enema bottle, or resuspended from a dry powder to form a solution for enteral administration.

The present invention is further understood by reference to the following non-limiting examples.

### EXAMPLES

### Example 1

### Coated Budesonide nanoparticles for Enhanced Penetration of GI Mucosa

### Materials and Methods

### Materials

Budesonide (99% purity), 5% w/v 2,4,6-trinitrobenzenesulfonic acid (TNBS) solution, sodium azide, dimethyl sulfoxide (DMSO), 1,4-Dithiothreitol (DTT), phosphate buffered saline (PBS), lipopolysaccharide (LPS), collagenase type VIII, and DNAse-I were purchased from Sigma-Aldrich (St. Louis, Mo.). Polyvinylpyrrolidone K30 (PVP) was purchased from TCI Chemicals (Portland, Oreg.). Zirconium oxide beads (0.15 mm, 0.5 mm and 1.0 mm in diameter) were purchased from Next Advance (Averill Park, N.Y.). Carboxylate-functionalized fluorescent polystyrene (PS) particles (200 nm red and 2 μm yellow-green) were purchased from Molecular Probes (Eugene, Oreg.). Sterile cell strainers (100 μm) were purchased from Fisher Scientific (Hampton, N.H.). Pluronic F127 (Poloxamer 407) was obtained as a free sample from BASF corporation (Tarrytown, N.Y.). Brefeldin A and fluorescent antibodies were obtained from eBioscience (San Diego, Calif.) or Biolegend (San Diego, Calif.).

### 2,4,6-trinitrobenzenesulfonic acid (TNBS) model

All experimental procedures were approved by the Johns Hopkins Animal Care and Use Committee. Male 5-6 week old Balb/c mice were purchased from Harlan (Indianapolis, Ind.) and acclimated in the animal facility for one week. Reducing the presence and passage of pellets during TNBS model induction minimizes variability in disease severity. Mice were starved for 6 h and then received a 200 μL saline enema using a flexible feeding tube to clear the distal 3-4 cm of the colorectum. After 10 min, 65 μL of TNBS (2.5% TNBS in 50% ethanol) was administered colorectally using a Wiretrol with the mice under isoflurane anesthesia to minimize back flow (Day 0, D0). These procedures resulted in reliable weight reduction (average ˜10-12% for each experimental group) within 24 h after TNBS administration (Day 1, D1). The few mice that did not exhibit at least 7% weight reduction 24 h after TNBS administration were excluded, and the remaining mice were grouped to ensure similar average weight loss.

### Model Particle Distribution

Stock (2% w/v) fluorescent PS particles were diluted 10-fold with 2% (w/v) Pluronic F127 solution in water (200 nm/2% F127) or 1% (w/v) PVP solution in water (2 μm/1% PVP) and stored in the fridge overnight. Mice in the “healthy” group did not receive any pretreatments. For the “TNBS” group, TNBS colitis was induced as described above (average weight loss μ10%). Fifty μl of particles was administered intra-rectally. After 15 min, the distal 4 cm of the colon was excised and cut into 1 cm portions that were embedded, frozen, sectioned, and stained as described. The sections were imaged at different magnifications using a Zeiss LSM 510 Meta confocal microscope.

### Budesonide Particle Formulation and Characterization

For the microsuspension (MS), budesonide (5 mg) and 1 ml of 1% (w/v) PVP solution in water were vortexed at 1200 rpm for 5 min. The particle size distribution was evaluated using a Beckman Coulter Multisizer IIe. For the nanosuspension, wet bead-milling was performed using a TissueLyser LT (Qiagen Inc, Germantown, Md). Budesonide (10 mg) and zirconium oxide beads were added to 1 ml of 2% (w/v) Pluronic F127 milled at a speed of 3000 oscillations per min in a cold room to dissipate heat. The milling beads were then separated by passing the suspension through a 100 μm cell strainer. The diameter of zirconium oxide beads, the mass ratio of beads to drug, and the bead-milling time were optimized to obtain a budesonide particle size of ˜200 nm. Particle size, polydispersity index (PDI), and surface charge (ζ-potential) of NS formulations were measured using a Malvern Zetasizer Nano ZS (173° scattering angle). NS were diluted 1:40 in 10 mM NaCl (pH 7) for ζ-potential measurements. Images of the NS were obtained using a Hitachi H7600 transmission electron microscope.

### Evaluation of In Vitro Activity

Mesenteric lymph nodes (MLN) and spleen were isolated from 7 week old C57BL/6 male mice. Single-cell suspensions of MLN and splenocytes were prepared as described. 200,000 MLN cells or splenocytes/well were seeded in 96 plate wells and activated with phorbol 12-myristate 13-acetate (PMA) (50 ng/ml), ionomycin (500 ng/ml), and LPS (1 μg/ml) for 5 h in the presence or absence of 1 μM of budesonide MS or NS. Brefeldin A was added to each well after 1 h of activation in order to enhance the detection of intracellular cytokines by flow cytometry. RAW 264.7 (murine macrophage cell line) cells were cultured at 37° C. in humidified atmosphere with 5% CO₂ using Dulbecco's Modified Eagle's Medium (DMEM) supplemented with 10% (v/v) heat-inactivated FBS, 100 U/ml penicillin, and 100 μg/ml streptomycin. For all experiments, 0.5 million cells/well were seeded overnight in 6-well plates. Then, the medium was replaced and cells were incubated for 5 h with 500 ng/ml LPS concomitantly with or without 1 μM of budesonide solution (0.02% DMSO in PBS), 1 μM budesonide MS, or 1 μM budesonide NS. Then, supernatants were removed, cells were washed with PBS and transferred into 96-well plates. Brefeldin A was added for 3 h and the cells were stained with relevant antibodies using flow cytometer (Table 1).

TABLE 1
Details of antibodies used for flow cytometry analyses.

| Cell type | Antibodies |
|---|---|
| RAW264.7 cell line | Surface staining: Anti-mouse CD16/CD32 FC blocker following by anti- |
| | mouse/human CD11b-PE antibody (Biolegend). |
| | Intracellular staining: Anti-mouse IL-1β-pro-form eFluor 450 antibody |
| | (eBioscience). |
| MLN and spleen | Surface staining: Anti-mouse CD16/CD32 FC blocker following by anti- |
| cells | mouse/human CD11b-PE antibody and anti-mouse CD4-APC (Biolegend). |
| | Intracellular staining: Anti-mouse TNFα-PE/Cy7 and anti-mouse IFNγ-PE |
| | (Biolegend). |
| Colonic LP cells | Surface staining: Fixable Viability Dye eFluor 520 (eBioscience) following |
| | by Anti-mouse CD16/CD32 FC blocker and anti-mouse CD45-PE/Cy7, |
| | anti-mouse/human CD11b-PE, anti-mouse Ly-6C-Pacific Blue, anti-mouse |
| | Ly-6G-PerCP/Cy5.5, anti-mouse CD4-APC, anti-mouse CD8-Brilliant |
| | Violet 421 and anti-mouse CD19-PE (Biolegend). |
| | Intracellular staining: Anti-mouse IL-1 beta Pro-form eFluor ® 450 |
| | (eBioscience). |

### Efficacy Experiment

TNBS colitis was induced as described above. Starting on D1, mice received a 100 μL enema containing either 0.5 mg/mL budesonide NS, 0.5 mg/mL budesonide MS, or PBS (untreated control). On Day 2 (D2) and Day 3 (D3), mice were weighed and given enema treatments for a total of 3 daily treatments. Twenty-four hours after the final treatment (Day 4, D4), mice were weighed, and colorectal tissues were obtained. A standard length of 4 cm of colorectal tissue were obtained from each mouse and weighed. Alternatively, the whole colon was used for histology or for isolation of immune cells from the lamina propria (LP).

### Histology

Whole colons were prepared using a “sandwich roll” technique, fixed in 10% neutral buffered formalin for at least 24 h, and then transferred to 70% ethanol for at least 24 h. JHU Reference Histology lab performed paraffin embedding, sectioning, and hematoxylin and eosin (H&E) staining. Tissues were scored by a trained veterinary pathologist in a blinded fashion using the scoring system in Table 2. The overall score was the sum of individual scores.

TABLE 2
Criterion for histological scoring

| Criterion | 0 | 1 | | 2 | 3 | |
|---|---|---|---|---|---|---|
| Infiltration | None | Increased number | | Inflammatory | Transmural | |
| | | of inflammatory | | cells extending | inflammatory cell | |
| | | cells in lamina | | into the submucosa | infiltration | |
| | | propria | | | | |
| Mucosal damage | None | Discrete | | Erosions or | Severe mucosal damage | |
| | | epithelial lesions | | focal ulcerations | with extensive ulceration | |
| | | | | | extending into the bowel | |
| | | | | | wall | |
| Atrophy and | Normal crypts | Mild | | Moderate | Severe | |
| crypt loss | | | | | | |
| Epithelial | Complete | Slight injury | | Surface not intact | No tissue repair | |
| regeneration | | | | | | |
| | 0 | 1 | 2 | 3 | 4 | 5 |
| Percent colon | 0% | ≤10% | 10-25% | 25-50% | 50-75% | 75-100% |
| involved | | | | | | |

### Isolation of Colon Lamina Propia (LP) Cells

Colonic LP cells were isolated as described previously, counted, and stained with relevant antibodies for flow cytometry analysis (Table 1). For intracellular staining, LP cells were activated with PMA (50 ng/ml) and lonomycin (500 ng/ml) for 4 h. Brefeldin A was added after 1 h of activation.

### Flow Cytometry Analysis

The antibodies used to detect relevant immune cells and intracellular cytokines from RAW264.7 cells, MLN/spleen cells and colonic LP cells are described in Table 1. Viability, surface and Intracellular staining were performed according to regular standard protocols established by eBioscience (San Diego, Calif.). All cells were analyzed using a SONY SH800 Cell sorter and FlowJo software.

### Statistics

The Student's t-test (two-tailed, unequal variance) was used to compare differences between two experimental groups. To compare multiple groups, one-way ANOVA with the Tukey post hoc test was used. For histological scoring, the non-parametric Kruskal-Wallis with Dunn's post hoc test was used. All values are presented as the average±the standard error of the mean (SEM) unless otherwise indicated. P values <0.05 were considered statistically significant for all data.

### Results

### Colorectal Distribution of Model Fluorescent Particles

Here, since the budesonide nano- and microsuspensions were composed only of budesonide and a physically associated surface coating material, it would be technically challenging to fluorescently label a component of the formulations without significantly impacting the stability and/or the physicochemical properties of the formulations. Thus, fluorescent polystyrene particles were used with the same respective surface coatings as model probes for assessing particle distribution and accumulation in the inflamed colons of mice with TNBS-induced colitis. Improved epithelial distribution and penetration of inflamed mucosa with the 200 nm/2% F127 particles was observed compared to the 2 μm/1% PVP particles, which is likely due to both the smaller size and the muco-inert surface coating. The use of PVP as a surface coating for the MS was to mimic the clinical budesonide formulation, though the results here do not directly assess the impact of using PVP as a surface coating compared to Pluronic F127.

Fluorescent PS particles of the appropriate size and surface coatings (Table 3) to serve as model probes for the budesonide NS and MS were intrarectally administered to both healthy mice and mice with TNBS-induced colitis. The lack of colorectal folds and presence of epithelial damage was immediately apparent in the mice with TNBS-induced colitis. Despite this, the 2 μm/1% PVP particles were largely dispersed in the lumen in the TNBS mice, similarly restricted from reaching much of the epithelium as in the colons of healthy mice In contrast, 200 nm/2% F127 particles distributed throughout the colorectal lumen in the TNBS mice, including penetrating into ulcerated regions of the tissue. In healthy mice, 200 nm/2% F127 particles were more uniformly distributed across the colorectal epithelium, as was previously demonstrated for similarly sized PS nanoparticles with covalently grafted polyethylene glycol (PEG) on the surface administered in a hypotonic enema.

### Development and Characterization of Budesonide MS and NS

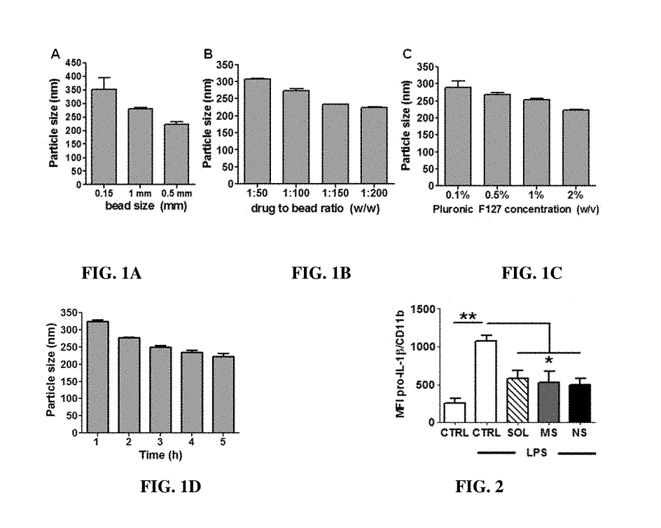

Budesonide MS were formulated using the same stabilizer in Entocort®, PVP. The average particle size was 2.5±1.25 μm. For the budesonide NS, various parameters involved in the wet-milling process using the Tissuelyser were optimized, including milling bead size (FIG. 1A), the ratio of mass of drug to mass of beads (FIG. 1B), the concentration of Pluronic F127 (FIG. 1C) and milling time (FIG. 1D) to obtain an F127-coated budesonide NS formulation with the appropriate size and surface properties for effective penetration through colorectal mucus (Table 3). Transmission electron micrograph (TEM) micrograph of budesonide NS further confirmed the size of budesonide NS.

TABLE 3
Summary of particle size, polydispersity and zeta potential data
of different budesonide formulations and surrogate particles

| Particle | | | Polydispersity | | |
|---|---|---|---|---|---|
| formulation | Particle size | | index | Zeta potential | |
| 2 μm PS/1% PVP | 1.8 ± 0.1 | μm | Not determined | −17 ± 2 | mV |
| 200 nm PS/2% | 235 ± 3 | nm | 0.07 ± 0.06 | −4 ± 0.2 | mV |
| F127 | | | | | |
| Budesonide MS | 2.5 ± 1.3 | μm | Not determined | −5 ± 0.3 | mV |
| Budesonide NS | 222 ± 9 | nm | 0.09 ± 0.03 | −2 ± 0.1 | mV |

### In Vitro Anti-Inflammatory Activity of Budesonide MS and NS Using Murine MLN

In order to confirm that processing does not impact the inherent activity of budesonide, the in vitro anti-inflammatory activity of the budesonide solution (SOL), NS and MS using murine macrophages (RAW264.7 cells) was evaluated. Flow cytometry analysis showed that all budesonide treatments significantly reduced the pro-form of IL-1β in the LPS-activated RAW264.7 cells, indicating that budesonide SOL, MS as well as NS exerted similar anti-inflammatory effects in vitro (FIG. 2). Budesonide MS as well as NS significantly reduced the levels of inflammatory cytokines TNFα and IFNγ in activated MLN and splenocytes.

### Efficacy in the TNBS Colitis Model

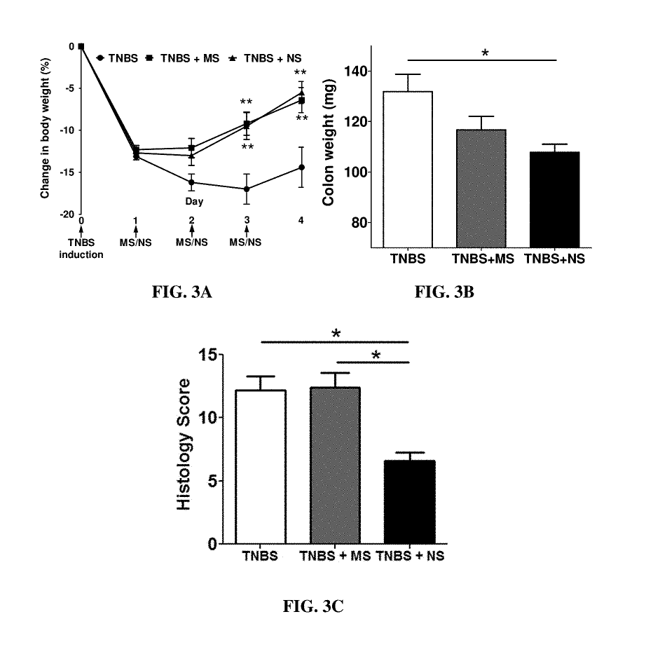

Daily budesonide enema treatment was evaluated for efficacy in an acute TNBS-induced colitis model. After the first enema treatment on D1, untreated mice (TNBS) continued to lose weight on D2 (−16.2±1%), whereas the average loss in body weight in the budesonide MS (TNBS+MS) and NS (TNBS+NS) group remained similar (−12.0±1.1% and −13.0±1.1% respectively). By D3, the average weight loss in the NS group (−9.5±1.6%) and MS group (−9.2±1.4 0%) was significantly less than the average weight loss in the control group (−17.0%±1.8%). Twenty-four hours after the third treatment on D4, the average weight loss for the NS (−5.5±1.3%) and MS (−6.4±1.5%) groups were similar, but significantly lower (P<0.01) compared to the untreated group (FIG. 3A). Importantly, although the overall recovery in weight loss was similar in the NS and MS groups, the average colon weight in the NS group (107.8±3.2 mg) was significantly decreased compared to the untreated group (131.9±6.9 mg), whereas the MS treated group was statistically indistinguishable compared to the untreated group (FIG. 3B). The improved local tissue effect of the budesonide NS was further confirmed with histopathological analysis of colon tissue. H&E stained colon sections revealed local alterations of cellular structure, ulcerations, neutrophil infiltration, and tissue damage in untreated control and MS treated mice, whereas treatment with the budesonide NS provided amelioration of microscopic IBD symptoms. Quantitative histopathological scoring based on mucosal damage, destruction of cellular architecture, infiltration and epithelial regeneration, and percent colon involved demonstrated a significant reduction in disease with budesonide NS enema treatment compared to untreated mice and MS treated mice (n=5 per group). Data are expressed as mean ±SEM; *p<0.05, **p<0.01 (FIG. 3C). Colorectal tissues from the untreated group showed classic signs of TNBS-induced colitis, such as infiltration of immune cells, loss of epithelial barrier, ulcerations, and atrophy and loss of crypt structure (FIG. 3C). Of note, only mice treated with the budesonide NS had a significant reduction in microscopic symptoms of IBD, whereas colorectal tissues from the mice treated with budesonide MS were indistinguishable from the untreated mice (FIG. 3C).

### Evaluation of Immune Cells

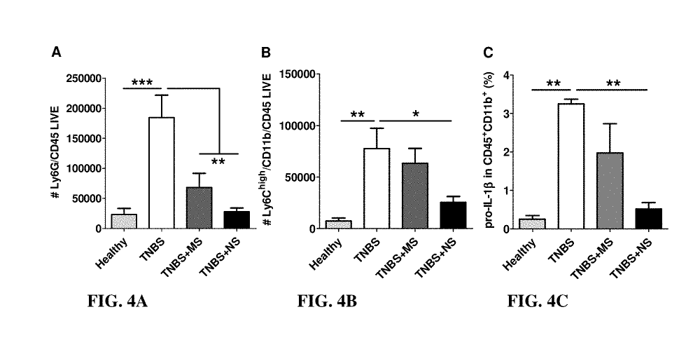

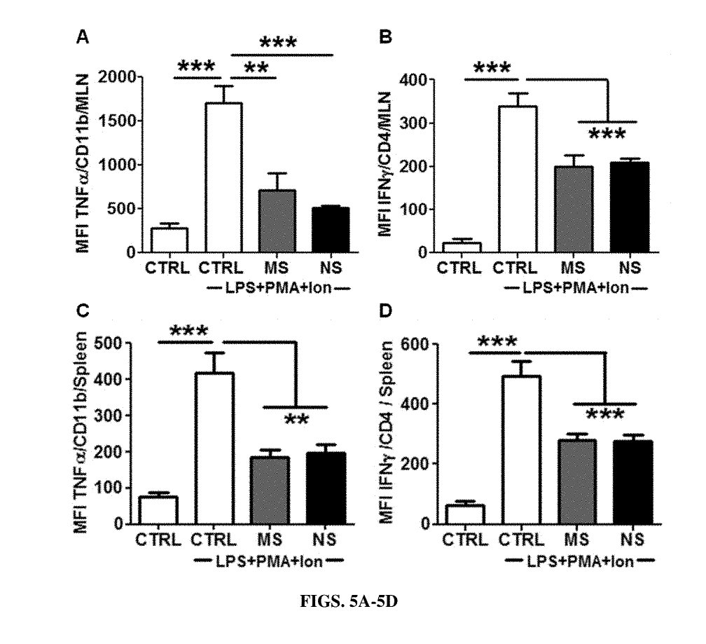

To further understand the increased efficacy of the budesonide NS in attenuating local colorectal inflammation, colon tissue immune cells were characterized. Viable colonic LP immune cells (CD45+ LIVE) were isolated and flow cytometry analysis was used to identify neutrophils (CD45+Ly6G+) and inflammatory macrophages (Ly6C^{high}CD11b+). Both the budesonide MS and NS treatments caused significant reductions (P<0.01) in the infiltration of neutrophils into the colon, though the NS trended toward the larger reduction (FIG. 4A). Only the budesonide NS treatment provided a significant reduction (P<0.05) in the infiltration of inflammatory macrophages that are involved in the progression of IBD (FIG. 4B) and significant reduction in the production of the pro-form of IL-1β from CD11b+ cells (FIG. 4C).

FIGS. 5A-5D are graphs showing in vitro anti-inflammatory activity of budesonide MS and NS. MLN cells and splenocytes were isolated from C57BL/6 mice and activated with LPS (1 μg/ml), phorbol myristyl acetate (PMA; 50 ng/ml) and ionomycin (Ion; 500 ng/ml) for 5 hours in the presence or absence of 1 μM of budesonide MS or NS and the mean fluorescence intensity (MFI) of (A) & (C) TNF-α gated on CD11b+ cells and (B) & (D) interferon-γ gated on CD4+ T cells was measured using flow cytometry. Budesonide MS and NS were able to reduce the MFI of inflammatory cytokines in activated MLN cells and splenocytes.

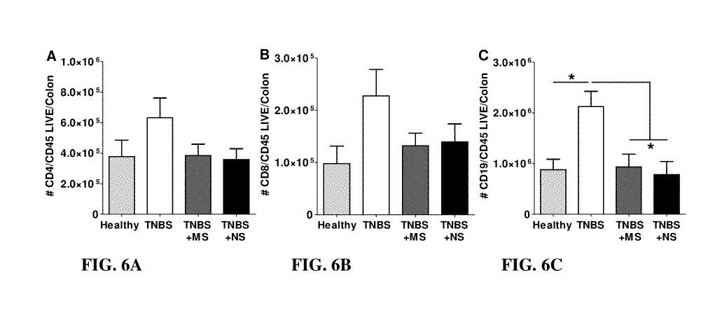

It was discovered that the budesonide MS and NS similarly reduced the presence of adaptive immune cells, including CD4+ T cells (FIG. 6A), CD+ T cells (FIG. 6B), and B cells (FIG. 6C).

### Discussion

The commonly used TNBS-induced colitis model was employed for comparing the efficacy of the budesonide NS and MS enema formulations. Of note, the TNBS model has more immunological similarities with human CD (Tₕ1 driven) than with ulcerative colitis (“UC”) (Tₕ2 driven), though in humans, inflammation and mucosal damage in CD is not restricted to the colorectum like in UC. However, the TNBS model is an attractive option for testing enema formulations, because the disease is reproducible and localized to the distal colorectum. Given that enemas are not expected to reach past the splenic flexure, enemas would not be expected to reach diffuse disease, particularly in the small intestine, which is more characteristic of CD. Furthermore, given the general anti-inflammatory properties of steroids like budesonide, it is not surprising that budesonide has been demonstrated to have efficacy in both the dextran sodium sulfate (DSS)-induced IBD model and the TNBS-induced models of IBD. However, given the diffused nature of the damage induced by orally administered DSS, the model is less optimal for testing enema formulations. To compare the muco-inert NS formulation to a clinically-relevant budesonide MS, the TNBS model was appropriate for demonstrating improved efficacy for the NS. The studies using the TNBS-induced colitis model showed that although both budesonide formulations led to a recovery in weight loss, the budesonide NS was more effective in reducing colon inflammation and normalizing tissue weight. Better distribution and penetration of muco-inert budesonide NS into the inflamed colon tissue could be responsible for the improved local effect. Histopathological studies also showed that only the mucus-penetrating budesonide NS enema could significantly ameliorate microscopic symptoms of IBD.

The efficacy of budesonide MS and NS formulations was also tested by analyzing immune cells in the colonic LP obtained from treated mice. Indeed, it was discovered that local administration of both budesonide MS and NS significantly reduced the absolute number of viable colonic CD45+Ly6G+ cells (neutrophils) in mice with TNBS-induced colitis. Glucocorticoids like budesonide can promote the resolution of inflammation, and restore homeostasis, by promoting a wound-healing and anti-inflammatory activity in macrophages, inducing neutrophil and T cell apoptosis and promoting the removal of apoptotic cells. Moreover, it has been shown that glucocorticoids can induce the expression and secretion of Annexin-1, which can induce apoptosis of neutrophils at the site of inflammation. Thus, the reduction in neutrophils observed with budesonide MS or NS treatment could be a result of the apoptotic effect of glucocorticoids such as budesonide on neutrophils in the inflamed colon. Macrophages have been implicated in the pathogenesis of a variety of chronic and autoimmune diseases, including IBD. The classically activated macrophages are key producers of many cytokines (e.g. IL-1β, TNFα and IL-6) and reactive metabolites of oxygen and nitrogen (e.g. nitric oxide) that have been implicated in the development of IBD. Macrophages, together with neutrophils, may contribute to intestinal damage by releasing reactive metabolites of oxygen and nitrogen. It was previously shown that Ly6C^{high} monocytes are recruited into the inflamed gut to become the dominant inflammatory mononuclear cell type in the LP during acute colitis. It was discovered that only budesonide NS enema treatment could significantly reduce the number of viable colonic infiltrating monocytes (Ly6C^{high}CD11b+) in mice with TNBS-induced colitis, and to significantly reduce the production of inflammatory cytokines from CD11b+ cells. It is likely that the increased effect of the budesonide NS on reducing local inflammation led to the significant improvement in tissue histology observed in this acute TNBS-induced colitis model.

Finally, the mucus-penetrating budesonide NS enema formulation is composed of all FDA GRAS ingredients without chemical modification.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A nanoparticle formulation for drug delivery to the gastrointestinal epithelium through the mucosa, the nanoparticles formed of the therapeutic or prophylactic agent for treatment of a colonic disease or disorder, optionally including a diagnostic agent, and having a coating of a mucus penetrating polymer thereon in an amount effective to facilitate passage through the mucosa of the colon.

2. The nanoparticle formulation of claim 1 wherein the mucus penetrating polymer comprises a polyalkylene oxide.

3. The nanoparticle formulation of claim 2 wherein the polyalkylene oxide is a polyalkylene glycol.

4. The nanoparticle formulation of claim 3 wherein the polyoxyethylene polyalkylene oxide block copolymer (Poloxamer).

5. The nanoparticle formulation of claim 4 wherein the Poloxamer is F127.

6. The nanoparticle formulation of claim 1 wherein the nanoparticles have an average dimension between 150 and 600, between 200 to 450, or between 200 and 300 nm.

7. The nanoparticle formulation of claim 6 wherein the population has an average diameter of approximately 200 to 250 nm.

8. The nanoparticle formulation of claim 1 coated with polyoxyethylene polyalkylene oxide block copolymer (Poloxamer F127) and has a negative surface charge, such as −2 mV.

9. The nanoparticle formulation of claim 1 wherein the agent is for treatment of colitis or inflammatory bowel disease.

10. The nanoparticle formulation of claim 9 wherein the agent is an anti-inflammatory.

11. The nanoparticle formulation of claim 10 wherein the agent is budesonide.

12. The nanoparticle formulation of claim 1 comprising a hypotonic vehicle for increased uptake in the colon.

13. A method of treating an individual with a colonic disease or disorder comprising administering the formulation of claim 1 to an individual in need thereof.

14. The method of claim 13 wherein the formulation is in the form of an enema.

15. The method of claim 13 wherein the individual has ulcerative colitis, diverticulitis, Crohn's disease or inflammatory bowel disease.

16. The method of claim 13 wherein the individual has diarrhea, hemorrhoids, or lactose intolerance.

17. The method of claim 13 wherein the individual has been treated for cancer with a chemotherapeutic.
